(19) United States
(12) Patent Application Publication
(10) Pub. No.: US 2011/0065104 A1
(43) Pub. Date: Mar. 17, 2011

(54) REAL-TIME ASSAYS OF NEURO-HUMORAL FACTORS TO ASSESS CARDIOVASCULAR STRESS

(76) Inventors: Donald W. Landry, New York, NY (US); Juan Oliver, New York, NY (US); Milan N. Stojanovic, Fort Lee, NJ (US)

(21) Appl. No.: 12/765,607

(22) Filed: Apr. 22, 2010

Related U.S. Application Data
(63) Continuation of application No. 12/144,760, filed on Jun. 24, 2008.

(51) Int. Cl. C12Q 1/68; G01N 33/543

(52) U.S. Cl. 435/6; 436/501

(57) ABSTRACT

The present invention relates to rapid assays for neuro-humoral factors modulated in response to cardiovascular stress and integration of data obtained from such assays to provide profiles of response to cardiovascular stress that can guide therapy.

### CROSS-REFERENCE TO RELATED PRIORITY

The present application claims priority to U.S. Provisional Application Ser. No. 60/755,311 filed on Dec. 30, 2005, the contents of which is incorporated herein in its entirety.

### GRANT INFORMATION

The subject matter of this application was developed at least in part using funds from a grant from the Long Range Navy and Marine Corps Science and Technology Program BAA, so that the United States Government has certain rights herein.

### 1. INTRODUCTION

The present invention relates to rapid assays for neuro-humoral factors modulated in response to cardiovascular stress and integration of data obtained from such assays to provide profiles of response to cardiovascular stress that can guide therapy.

### 2. BACKGROUND OF THE INVENTION

Hemorrhage is characterized by a decline in cardiac output and by a compensatory vasoconstriction that supports perfusion of vital organs. Vasoconstriction worsens peripheral tissue perfusion in hemorrhagic shock and the goal of early resuscitation is to staunch bleeding and replenish intravascular volume and, thereby, restore cardiac output and perfusion.

However, severe hemorrhagic shock, particularly if resuscitation is delayed or protracted, can result in a transition to a late phase unresponsive to the restoration of intravascular volume. Late-phase hemorrhagic shock is a form of vasodilatory circulatory collapse previously thought to represent a pre-terminal failure of vascular smooth muscle cells. It has been discovered, however, that late-phase hemorrhagic shock does not reflect a general collapse of the contractile machinery of vascular smooth muscle, but rather reflects the deficiency of a hormone, vasopressin (see, for example, Landry and Oliver, 2001, N. Engl. J, Med. 345:588-595; Holmes et al., 2003, Critical Care 7:427-434; and Holmes et al., 2004, Critical Care 8:15-23).

In early hemorrhagic shock, vasopressin is markedly elevated but, in the late phase, plasma levels have declined significantly. For example, in a study of hemorrhagic shock in dog (Morales, 1999, Circulation 100(3):226-229) plasma vasopressin concentration averaged over 300 pg/mL (normal <5 pg/mL) during the acute phase of severe blood loss, but declined to levels below 30 pg/mL after 60-90 minutes of sustained and severe low blood pressure. Inappropriately low vasopressin in hemorrhagic shock is of interest because administration of low doses of the hormone (˜0.04 U/60 Kg/min), which increase its plasma concentration to levels similar to those found during the initial phase of shock (˜50-300 pg/mL), significantly increase arterial pressure (˜25-50 mm Hg). Vasopressin is therefore Likely to be an essential component in the armamentarium of treatments for the vasodilatory shock associated with the late-phase of hemorrhagic shock.

The cardiovascular system evolved to achieve the convective transport of nutrients and oxygen to distant tissues and achieve removal of waste products and carbon dioxide. Blood pressure drives the flow in the transport system. But blood pressure in itself is a complex function of cardiac output, systemic vascular resistance, and blood viscosity, among other factors. A variety of neuro-humoral factors, in addition to vasopressin, regulate the determinants of blood pressure, including norepinephrine, epinephrine, renin, endothelin peptides, atrial natriuretic peptide (“ANP”), and brain natriuretic peptide (“BNP”). Clearly, marked derangements in blood pressure (e.g., the blood pressure of shock states) signify that an organism is succumbing to a cardiovascular stress. It is difficult, however, for a clinician to discern what patients are about to go into shock because a normal blood pressure may precede a catastrophic drop in blood pressure. In such circumstances, normal blood pressure may represent a metastable state maintained through the activation of neuro-humoral factors that may precipitously evolve to a formal collapse of function.

Because changes in levels of neuro-humoral factors may be harbingers of impending shock, the ability to assay such factors rapidly and in an ongoing fashion would provide a valuable guide for diagnosis, treatment, and prognosis of patients under cardiovascular stress not yet apparent through a discernable change in blood pressure. However, no system currently available can provide this information in a timely fashion, and permit an integration of levels of these factors into an appropriate assessment of disease.

### 3. SUMMARY OF THE INVENTION

The present invention relates to assays for neuro-humoral factors (“NHFs”) that may be performed in “real time,” meaning a time frame that permits the utilization of at least one NHF level as an index of hemodynamic stability and as the basis for clinical decision making in an acute-care context. The assays of the invention are directed to improving NHF capture, either through aptamers, including heteromeric DNA aptamers and/or high affinity antibodies.

### 4. BRIEF DESCRIPTION OF THE DRAWINGS

FIG. 1A-C. (A) Vasopressin and its components, (B) pressinoic acid (amino acids 1-6 of arginine vasopressin) and (C) gly-7-9, representing the remainder of the molecule.

FIG. 2. Generation of bivalent heterodimeric aptamers. The peptide (WE) is split into two hemispheres (W west and E east), which are individually attached to agarose affinity matrices. Using in vitro selection and amplification, individual aptamers binding to W and E fragments are isolated and afterwards combined.

FIG. 3A-C. Affinity column for rapid purification of vasopressin. (A) Aptamer is attached to the affinity matrix (e.g. agarose); serum samples are passed over this material, leading to the binding and concentration of vasopressin to agarose, and extensive washing eliminates interfering materials. (B) Aptamer may be cleaved off the column together with vasopressin, if a cleavable linker is used (e.g. diol with sodium periodate, or ester or amide cleaved with appropriate esterase or protease); if the aptamer is fluorescently labeled, the cleaved material could be directly read out. (C) Vasopressin is cleaved off with denaturing conditions, for example, with ethanol; upon evaporation of ethanol, fluorescent sensors could be added. Note that the preconcentration of vasopressin can lead to a significant increase in sensitivity of this assay.

FIG. 4A-D. Four representative sensor designs. (A) Monoflurorophoric sensors (Jhaveri et al., 2000, J. Am. Chem. Soc. 122:2469-2473; Jhaveri et al., 2000, Nature Biotechnol. 18(12):1293-1297; Stojanovic et al., 2003, J. Am. Chem. Soc. 125:6085-6089); Fluorophore is displaced by vasopressin in a G-poor environment resulting in an increase in fluorescence; Alternatively, sensor tumbling is reduced resulting in a change in fluorescence polarization. (B) Bisfluorophoric sensors (Stojanovic et al., 2001, J. Am. Chem. Soc. 123(21):4928-4931); a change in distance between fluorescence donor and acceptor (or quencher) results in the fluorescence signal. (C) Self-assembling sensors; heterodimer is stabilized in the presence of vasopressin, leading to a change (usually a decrease) in fluorescence signal. (D) Dye (luminescent in a complex with DNA) is displaced by vasopressin (Stojanovic and Landry, 2002, J. Am. Chem. Soc. 124:9678-9679), leading to decrease in luminescent signal.

FIG. 5. Basic principle behind the proximity ligation assays. The target peptide is used to organize two aptamers with extended oligonucleotide sequences that can serve as substrates for a ligase. Upon ligation, real-time PCR is used to determine the concentration of the peptide, with possibilities for multicolor detection.

### 5. DETAILED DESCRIPTION OF THE INVENTION

For purposes of clarity, and not by way of limitation, the detailed description of the invention is divided into the following subsections:

(i) the preparation of NHF-binding aptamers;

(ii) heteromeric aptamers;

(iii) anti-NHF antibodies;

(iv) assay systems;

(v) diagnostic uses of the invention.

A compound is a NHF, as defined herein, if it is a factor that is associated with blood pressure. Non-limiting examples of NHFs include vasopressin, norepinephrine, epinephrine, renin, endothelin peptides, atrial natriuretic peptide (“ANP”), and brain natriuretic peptide (“BNP”). Description herein applied to vasopressin may be analogously applied in assays for other NHF molecules. An “aptamer” is a nucleic acid molecule, which in specific non-limiting embodiments is more than 5 or 10 and less than 100 (which means, more than 5 and less than 100, or, more than 10 and less than 100), or more than 5 or 10 and less than 80, or more than 5 or 10 and less than 60, or more than 5 or 10 and less than 50, or more than 5 or 10 and less than 30, nucleotides in length, comprising deoxyribonucleotides and/or ribonucleotides.

### 5.1 The Preparation of NHF-Binding Aptamers

NHF-binding aptamers may be prepared and/or identified using technology as developed for aptamer selection described, for example, in Ellington and Szostak, 1990, Nature 346:818-822; German et al., 1998, Anal. Chem. 70:4540-4545; Jayasena, 1999, Clinical Chem. 45(9):1628-1650; Jhaveri et al., 2000, Nature Biotechnol. 18(12):1293-1297; Nutio et al., 2003, J. Am. Chem. Soc. 125:4771-4778; Stojanovic et al., 2003, J. Am. Chem. Soc. 125:6085-6089; Tuerk and Gold, 1990, Science 249:505-510; Wang et al., Biochemistry 35:12338-12346; Michaud et al., 2003, J. Am. Chem. Soc. 125(28):8672-8679; Wilson and Szostak, 1999, Annual Rev. Biochem. 68:611-647; or Williams et al., 1997, Proc. Natl. Acad. Sci. U.S.A. 94:11285-11290.

In non-limiting examples, Systematic Evolution of Ligands by EXponential enrichment (“SELEX”) may be used (see, for example, Kopylov and Spiridonova, 2000, Molecular Biology 3416):940-954, translated from Kopylov and Spiridonova, 2000, Molekulyarnaya Biologiya 34(6):1097-1113). A synthetic oligonucleotide library may be screened for binding to a NHF target (see below), bound aptamers may be eluted and amplified by polymerase chain reaction (“PCR”), and one or more additional binding steps may be performed to obtain selectively tighter binding aptamers.

A NHF target, as the term is used herein, may be (i) an intact NHF molecule, (ii) a fragment of a NHF molecule where, if the NHF is a protein, the fragment contains at least 10 percent, 20 percent, 30 percent, 40 percent, 50 percent, 60 percent, 70 percent, 80 percent, or 90 percent of the intact molecule (biologic activity of the NHF need not be retained but desirably binding of the aptamer to the fragment correlates with binding of the aptamer to intact NHF); or (iii) an intact NHF or a NHF fragment bound to a carrier molecule (other than a carrier molecule which is part of a purification matrix), for example where the carrier molecule may be a detectable label or may be a stabilizing peptide. Any of the above-listed NHF targets may further be bound to a molecule or structure which is part of a matrix, such as an affinity matrix used for selection of aptamer or for assay purposes.

In particular non-limiting embodiments of the invention, two or more different NHF fragments are each used to select a binding nucleic acid (DNA or RNA) aptamer, and the two or more resulting selected aptamers are joined, optionally via a linking molecule, to form a heterodimer or heteromultimer (generically referred to herein as “heteromers” or as being “heteromeric”).

In particular, non-limiting embodiments of the invention, the monomeric aptamers selected pursuant to this section may have a binding affinity for the corresponding NHF target of between about 100 nM<Kd<10 μM.

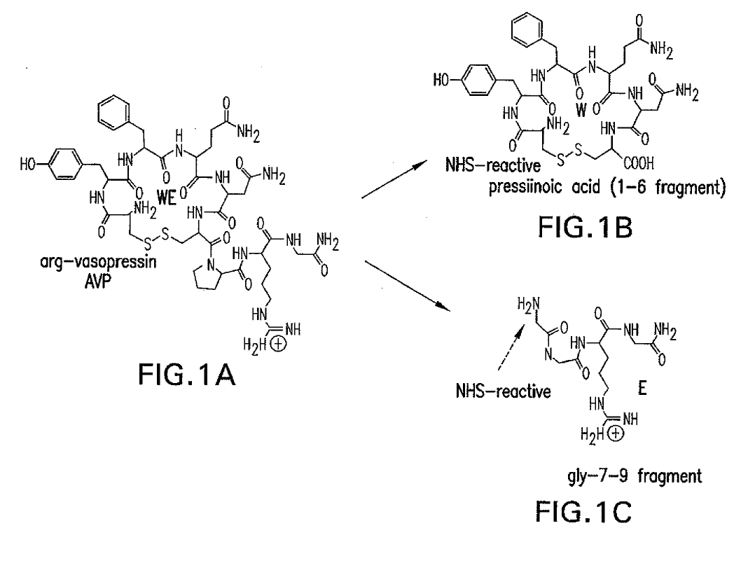

As a specific, non-limiting example of the invention, two different aptamers that bind to arginine vasopressin (“vasopressin”) may be prepared as follows. As it is unlikely that two aptamers against vasopressin targeting different binding sites (“epitopes”) of vasopressin could be isolated using the intact vasopressin molecule, because of the strong possibility of an epitope dominance effect, the vasopressin molecule may be divided into two fragments and one or more aptamer that binds to each respective fragment may be prepared. For example, one fragment may be the commercially available cyclic peptide pressinoic acid (W), and the other a gly-7-9 fragment (E) (see FIG. 1). To generate the affinity columns, the W fragment may be attached to Affigel-10 agarose gel (NHS-activated, Biorad) in bicarbonate buffer pH=8.5 diluted with glycine to afford a 200 μM concentration of pressinoic acid on the resulting affinity matrix. The E fragment may be attached in the same fashion. Each of these affinity matrixes may be separately or conjointly subjected to two or more independent rounds of selection and amplification, for example, but not by way of limitation, using one common oligonucleotide library and one library specific for each of the affinity materials. The general structure of the libraries may be, as but one example, (5′-primer)-N40-(3′-primer) (the randomized region is 40-merit). Heat pre-equilibrated libraries (˜1 nmol, 10-14 members) may be exposed to individual affinity columns, and washed with 10 volumes of binding buffer (20 mM TRIS, pH=7.4, 140 mM NaCl, 1.2 mM MgCl2, 5 mM KCl, 2 mM CaCl2), followed by the three-volumes of binding buffer with added vasopressin (200 μM) for affinity elution. After determining the identity of binding regions through a re-randomization process, individual aptamers may optionally be minimized using standard procedures, eliminating sequences not participating in binding or not required for proper folding.

### 5.2 Heteromeric Aptamers

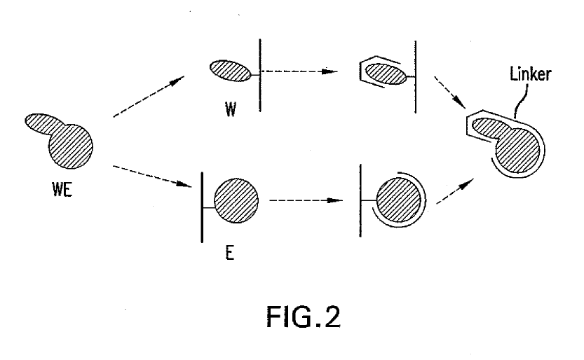

In order to provide for efficient capture of NHF, two or more aptamers, each having relatively lower affinity for a target NHF, may be joined, optionally via a linker, to produce a heteromeric aptamer with higher binding affinity than either of its component aptamers (see FIG. 2). In principle, for a heterodimer, assuming that the heterodimer allows complete accessibility to both binding sites, and where the dissociation constants for the individual aptamers are Kd1 and Kd2, the combined heterodimeric aptamer may exhibit a composite dissociation constant approximated by Kdc=Kd1*Kd2. In specific non-limiting embodiments, the higher affinity may be Kd<1 pM.

In non-limiting embodiments, a linker, if present, may be optimized using a further selection procedure, as follows. Two individual binding regions may be used as primers, and the linker region may be a N40 library (see above). A reselection process (under gel-shift assay conditions, with low pM concentrations of vasopressin) may be used to optimize the binding strength and determine the best spacer. This type of sensor may be amplified by PCR, and potentially useful for real time PCR as the method of detection.

In particular non-limiting embodiments, individual aptamers may be combined in a head-to-head (5′-5′), head-to-tail (5′-3′) or tail-to-tail (3″-3′) fashion. The head-to-tail heteromers may be directly ordered from custom-synthesis companies, with various number and lengths of internal polyethylene glycol spacers or (dT)n regions inserted between the monomeric regions. Alternatively, combinations may be chemically synthesized from monomers containing matching reactive functionalities; for example the 3′ (tail) of one monomer could be functionalized with a thiol, while the 5′ (head) of another could be derivatized with an acrylate moiety.

Individual constructs may be tested for binding to NHF (e.g., vasopressin), for example using gel shift assays with radiolabeled NHF, fluorescence polarization, surface plasmon resonance (e.g., using Biacore technology) with NHF attached to a chip, and/or equilibrium gel filtration with radioactive NHF. In specific non-limiting embodiments, a heteromeric aptamer having a binding constant below 200 pM may be tested for selectivity over similar peptides. In the event that the heteromeric aptamer shows significant cross-reactivity, a counter-selection procedure may be performed in order to achieve satisfactory selectivity. As regards vasopressin, heteromeric aptamers desirably demonstrate the potential to be responsive in low-to-mid picomolar range of vasopressin concentrations and show satisfactory selectivity. In specific, non-limiting embodiments, the invention provides for a heteromeric, especially a heterodimeric, aptamer comprising a vasopressin binding aptamer as disclosed in Williams et al., 1997, Proc. Natl. Acad. Sci. U.S.A. 94:11285-11290, and further comprising a second vasopressin binding aptamer, for example an aptamer selected using pressinoic acid (amino acids 1-6 of arginine vasopressin) or gly-7-9, as shown in FIG. 1, as NHF target.

### 5.3 Anti-NHF Antibodies

The present invention further provides for the use of anti-NHF antibodies for use as capture agents, alone or in combination with aptamers including heteromeric aptamers as set forth above. The term “antibodies” as used herein refers to intact immunoglobulin molecules, portions of immunoglobulin molecules retaining ligand-binding affinity, and single chain antibodies.

In particular, non-limiting embodiments of the invention, antibodies to vasopressin may be prepared as follows. Vasopressin may be attached to Keyhole Limpet Hemocyanin or other suitable carrier molecule at one three different sites, i.e., as pressinoic acid connected at the C-terminus through NHS chemistry, as vasopressin connected at its N terminus through an N-hydroxy succinimate ester, or as vasopressin connected at its tyrosine through diazo chemistry, to produce “vasopressin immunogen”. The vasopressin immunogen may be used to generate a panel of monoclonal antibodies which may bind to different epitopes of vasopressin. Such monoclonal antibodies may then be used as is or derivatized, fragmented, or engineered to produce useful capture agents.

### 5.4 Assay Systems

The present invention provides for NHF assay systems that utilize a capture agent which may be an aptamer e.g. a heteromeric aptamer, or antibody; in alternative embodiments more than one capture agent may be used. Although specific non-limiting examples provided below may relate to measurement of vasopressin, the assays described may be utilized to measure any NHF, using an aptamer that specifically binds to The NHF.

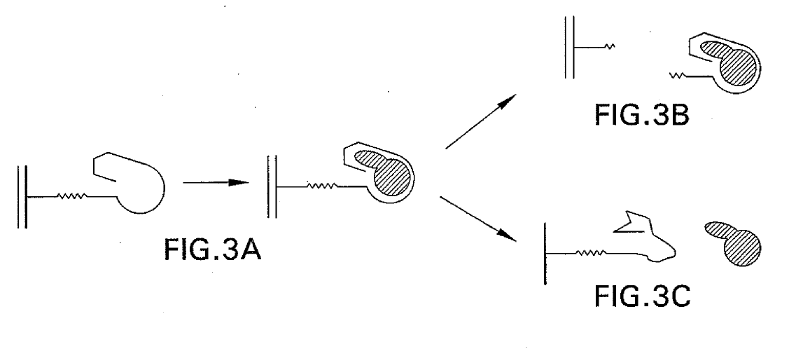

The assay may be performed using a sample obtained from a patient, including but not limited to a sample of blood or its components, urine, cerebrospinal fluid or tissue. The assay may be performed directly on the sample or indirectly, the latter meaning that the sample is processed prior to the assay. Non-limiting examples of processing include concentration of the sample itself (e.g., by centrifugation or filtration) or selective concentration of the NHF to be measured, for example using a chromatographic (e.g. SepPak C18 column) and/or an affinity method employing a capture agent such as an aptamer, heteromeric aptamer, or antibody. A schematic diagram of an affinity method is illustrated in FIG. 3. Both C18- and affinity-based purification may lead to slightly longer test times than direct assay, but would nevertheless significantly reduce them by eliminating prolonged plate incubations.

Real time PCR is an increasingly popular method for the detection of specific oligonucleotides in solution, and the concentration of aptamer may be coupled to the concentration of NHF (e.g., vasopressin). In specific non-limiting embodiments, aptamer or heteromeric aptamer may be added to a serum sample together with beads that would bind the aptamer tightly, where NHF and the bead would compete for aptamer binding. For example, the beads for the assay could be prepared (i.e., before exposure to patient sample) to comprise, at their surface, NHF (e.g., vasopressin) or an oligonucleotide complementary to at least a portion of the aptamer (the latter may provide the advantage of adjustable binding constants, and binding of aptamer to the bead takes it out of solution)). The NHF (e.g., vasopressin) in the serum sample would be expected to hinder the attachment of aptamer to beads, and the concentration of aptamer as determined through real time PCR would be directly proportional to the amount of NHF in the sample.

It should be noted that vasopressin in blood or plasma is subject to rapid degradation unless samples are collected in chilled containers and maintained at <4 C.

Aptamers and heteromeric aptamers as described above may be used in assays as set forth, for example, in Tombelli et al., 2005, Biosensors and Bioelectronics 20:2424-2434.

In one set of non-limiting embodiments, aptamers including heteromeric aptamers as described above may be used in chromatographic assay methods. As one specific, non-limiting example, a heterodimeric aptamer that binds to vasopressin may be linked to a support matrix; a sample, such as a patient serum sample, may then be exposed to the matrix so that vasopressin is retained on the matrix. The amount of vasopressin present may then be detected, for example using a method in which delectably (radioactively, fluorescently, etc.) labeled anti-vasopressin antibody is used to measure the amount of vasopressin bound to the matrix. Alternatively, detectably (radioactively, fluorescently, etc.) labeled vasopressin may be pre-bound to aptamer such as heteromeric aptamer linked to matrix, so that when sample is applied to the matrix, the labeled vasopressin may be displaced and eluted. The amount of labeled vasopressin either retained on the matrix or found in the eluate may then be measured.

In another set of non-limiting embodiments, aptamers including heteromeric aptamers as described above may be used in capillary electrophoresis assay methods. As one specific, non-limiting example, a heterodimeric aptamer that binds to vasopressin may be used as an affinity probe in capillary electrophoresis-based quantitative assay for a NHF (see, as cited in Tombelli et al. supra, German et al., 1998, Anal. Chem. 70:4540-4545; Kotia et al., 2000, Anal. Chem. 72:827-831). According to such methods, free versus bound aptamers such as heteromeric aptamers may be electrophoretically separated due to changes in electrophoretic properties which arise upon binding. Such aptamers may also be used in non-equilibrium capillary electrophoresis of equilibrium mixtures (see, as cited in Tombelli et al., supra, Berezovski et al., 2003, Anal. Chem. 75:1382-1386). The amount of NHF in the sample would be directly proportional to the amount of bound aptamer and indirectly proportional to the amount of unbound aptamer.

In yet another set of non-limiting embodiments, aptamers including heteromeric aptamers as described above may be used as optical sensors, for example in an assay in which NHF, such as vasopressin, in a sample (e.g., serum sample) may be detected by its ability to displace detectably labeled NHF bound to aptamer linked to microspheres (e.g. silica microspheres) in a fiber-optic biosensor system (see, as cited in Tombelli et al., supra, Lee and Walt, 2000, Anal. Biochem. 282:142-146).

In still further non-limiting sets of embodiments, aptamers including heteromeric aptamers as described above may be used as cantilever-based biosensors or as “signaling aptamers”, as described in Tombelli et al., supra.

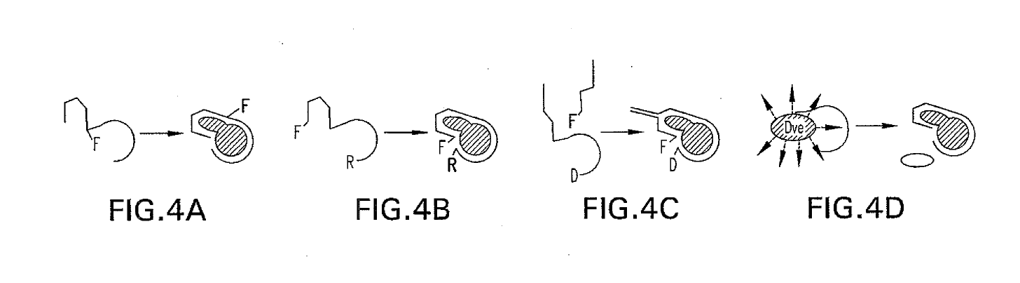

Homogenous assays based on heterodimeric aptamers. Mono- and bis-fluorophoric assays, dye displacement assays, in both competitive and non-competitive format, may be used to assay NHF, such as, but not limited to, vasopressin (see FIG. 4). Monofluorophoric fluorescent sensors may be generated from aptamers by systematic screening of fluorophore positions through substituting dT's with fluorescent dU analogs, coupled with testing for sensor activity. Competitive assays may utilize, for example, NHF-BODIPY-TMR vasopressin-BODIPY-TMR). For direct measurement of fluorescence, and without being bound by any particular theory, it is believed that these sensors primarily work through the displacement of dye from G-rich environment, in which they are severely quenched, to G-poor regions, in which quenching is less efficient. In principle, the bisfluorophoric sensors should have a much stronger response than monofluorophoric sensors. However, the ability to produce suitable bisfluorophoric sensors may depend on any conformational change(s) that aptamers undergo upon binding. Under some circumstances these conformational changes may be engineered. Displacement assays based on aptamers may be performed using techniques as in (Stojanovic and Landry, 2002, J. Am. Chem. Soc. 124:9678-9679), and displacement of luminescent metal complexes (e.g. [Ru(phen)2(dpzz)]2+, see Ossipov et al., 2001, J. Am. Chem. Soc. 123(15):3551-3562; Holmlin et al., 1998, Inorg. Chem. 37(1):29-34; Liu et al., 2005, J. Inorg. Chem. 99(12):2372-2380; Ambroise and Maiya, 2000, Inorg. Chem. 39(19):4256-4263; Holmlin et al., 1999, Bioconj. Chem. 10(6):1122-1130; Junicke et al., 2003, Proc. Natl. Acad. Sci. U.S.A. 100:3737-3742; Rube et al., 2004, Inorg. Chem. 43:4570-4578) from the double-helical regions of the aptamers by the binding of sample NHF (e.g., vasopressin) may be used.

Heterogenous assays. In certain non-limiting embodiments, the present invention provides for heterogenous assays based upon reported RIA and ELISA assays except that polyclonal antibodies would be substituted by a NHF such as vasopressin. This would allow heat-equilibration of samples in the presence of biotin-labeled aptameric receptors which may result in significant reduction of assay time.

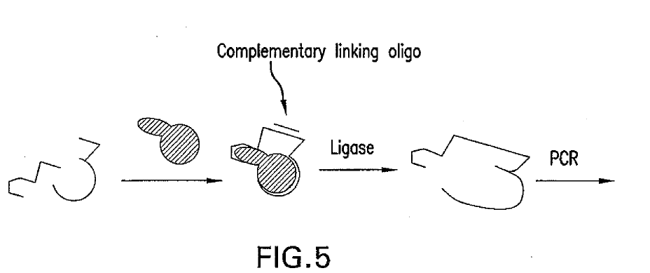

In other non-limiting embodiments, the present invention further provides for proximity ligation assays (see FIG. 5) using real-time PCR for detection of ligated products (where the amount of ligated product is directly proportional to the amount of NHF present. In still further non-limiting embodiments, the present invention provides for immunofluorescence assays in affinity probe capillary electrophoresis (APCE).

### 5.5 Diagnostic Uses of the Invention

The present invention uses the foregoing capture agents and assays to measure the level of NHF in an acute care setting. Detection of a change in NHF values from normal or acute shock levels may indicate that the patient is at risk for an imminent and catastrophic drop in blood pressure. In non-limiting embodiments of the invention, the change in NHF associated with a predictive value for a substantial decrease in blood pressure may be a change of at least about 20 percent, 30 percent, or 50 percent relative to normal or acute shock values for a particular NHF (including but not limited to published levels or levels in the patient or a suitable control)

As a specific, non-limiting example, in the case of vasopressin, the level of vasopressin associated with early shock in humans is approximately 22.7±2.2 pg/mL (Holmes et al., 2001, Chest 120:989-1002, citing Landry et al., 1997, Circulation 95:1122-1125), whereas the level of vasopressin associated with late shock is about 3.1±1.0 pg/mL for septic shock (Holmes et al., supra citing Landry et al., 1997, Circulation 95:1122-1125), <10 pg/mL after LVAD insertion (Holmes et al., supra, citing Argenziano et al., 1997, Circulation 96:II-286-290), 12.0±6.6 pg/mL after cardiopulmonary bypass (Holmes et al., supra, citing Argenziano et al. 1998, Thorac Cardiovasc. Surg. 116:973-980), median 3.3 pg/mL in children after cardiopulmonary bypass (Holmes et al., supra, citing Rosenzweig et al., 1999, Circulation 100:II 182-II 186), and 2.9±0.8 pg/mL in human organ donors (Holmes et al., supra, citing Chen et al. 1999, Circulation 100:II 244-II 246). Accordingly, the present invention provides, in non-limiting embodiments, for a method comprising using one or more capture agent, as set forth above, to measure the level of vasopressin in the serum of a subject, wherein a level below 20 pg/mL, or below 15 pg/mL, or below 10 pg/mL, or, especially for children, below 5 pg/mL, indicates that there is a substantial likelihood that the blood pressure of the patient is about to decrease to undesirable levels, or is already dangerously low and likely to be unresponsive to volume expansion. In specific non-limiting embodiments, an undesirable blood pressure is a systolic blood pressure of 90 mm Hg or less (or, in specific embodiments, 80 mm Hg or less, or 70 mm Hg or less) and/or a diastolic blood pressure of 60 mm Hg or less (or, in specific embodiments, 50 mm Hg or less, or 40 mm Hg or less). The present invention further provides for the additional step of inhibiting a decrease in blood pressure (meaning preventing or decreasing the magnitude of decrease) by administering a therapeutic agent, such as, but not limited to, low-dose continuous infusion of vasopressin or bolus administration of a partial agonist of the V1a receptor. In one specific non-limiting embodiment of the invention, a continuous low-dose infusion of between 0.01 and 0.04 U/min of vasopressin, for example until blood pressure is stabilized, may be administered.

### 6. REFERENCES

Amberg, G. C. (2003). “Modulation of the molecular composition of large conductance, Ca(2+) activated K(+) channels in vascular smooth muscle during hypertension.”. J Clin Invest 112(5): 717-24.
Argenziano M (1997). “A prospective randomized trial of arginine vasopressin in the treatment of vasodilatory shock after left ventricular assist device placement.”Circulation (Suppl II) 96:II-286-I-290
Bond, R. F (1967). “Cutaneous and skeletal muscle vascular responses to hemorrhage and irreversible shock.” Am J Physiol 212(2): 488-97.
Chan, W. Y. (2003). “Discovery and design of novel and selective vasopressin and oxytocin agonists and antagonists: the role of bioassays.” Exp. Physiology 85S:7s-18s
Chen J M (1999). “Vasopressin deficiency and pressor hypersensitivity in hemodynamically unstable organ donors.” Circulation (Suppl) 100:II244-II246
Cheema, F (2003). “Pressor effect of vasopressin in late-phase hemorrhagic shock is independent of inhibition of K⁺_{ATP} channel or the nitric oxide pathway.” ASN Abstracts 2003
Cook, D. B. (1995). “Monoclonal Antibodies in Diagnostic Immunoassays.” In Ritter, M. A., Ladyman, H. M. eds. Monoclonal Antibodies:180-208.
Cooke, C. R. (1993). “Reversible vasopressin deficiency in severe hypernatremia.” Am J Kidney Dis 22(1): 44-52.
Czernichow, P (1985) Clinical Chemistry 31:961.
Drolet, D. W (1996). “An enzyme-linked oligonucleotide immunoassay.” Nature Biotech. 14:1021-1025.
Drummond T. G. (2003). Electrochemical DNA sensors.” Nature Biotechnology 21:1192-1199.
Ellington A. D. (1990). “In vitro selection of RNA molecules that bind specific ligands.” Nature 346:818-822.
Evgenov, O. V. (2003). “Parenteral administration of glipizide sodium salt, an inhibitor of adenosine triphosphate-sensitive potassium channels, prolongs short-term survival after severe controlled hemorrhage in rats.” Crit Care Med 31(10): 2429-36.
Fredrikson, S. (2002). “Protein detection using proximity-dependent DNA ligation assays.” Nature Biotech. 20:473-477
German, I. (1998). “Aptamers as ligands in affinity probe capillary electrophoresis.” Anal. Chem. 70:4540-4545
Gold, J (2000) “Vasopressin in the Treatment of Milrone-induced Hypotension in Severe Heart Failure.” Am. J. Cardiol. 85:506-508
Holmes, C (2003). “Vasopressin and the cardiovascular system.” Critical Care 7:427-434
Jackson E. (2001). “Vasopressin and other agents affecting renal conservation of water.” The Pharmacological Basis of Therapeutics 10^{th} edition:789
Jackson, W. F. (2000). “Ion channels and vascular tone.” Hypertension 35(1 Pt 2): 173-8.
Jayasena, S. D. (1999) “Aptamers, an emerging class of molecules that rival antibodies in diagnostics.” Clinical Chemistry 45(9):1628-1650
Jhaveri, S (2000a). “Designed signalling aptamers that transducer molecular recognition to changes in flureoscence intensity.” J. Am. Chem. Soc. 122:2469-2473
Jhaveri, S (2000b). Nat. Biotech. 18(12):1293-97
LaRochelle, F. T. (1980). Pfluegers. Arch. 387:79
Landry D W (2001). “Mechanisms of disease: The pathogenesis of vasodilatory shock.” N Engl J Med 345:588-595
Landry D W (1997). “Vasopressin deficiency contributes to the vasodilation of septic shock.” Circulation 95:1122-1125
Landry, D W (1992). “The ATP-sensitive K+ channel mediates hypotension in endotoxemia and hypoxic lactic acidosis in dog.” J Clin Invest 89(6): 2071-4.
Lebl, M (1987). “Tables of analogs.” Handbook of Neurohypophyseal Hormone Analogs vol. II, part 2:127-267
Liu, J. J. (1994). “Human Internal Mammary Artery Responses to non-peptide vasopressin antagonists.” Clin. Exp. Pharm. Phys. 21:121-124
Lombard, J. H. (1980). “Prevention by methylprednisolone of norepinephrine depletion in vascular tissue during severe hemorrhage in dogs.” Blood Vessels 17(5): 276-80.
Mammen, M (1998). “Polyvalent interactions in biological systems: Implications for design and use of multivalent Ligands and inhibitors.” Angew. Chem. Int. Ed. 37:2754-2794
Nutiu, R (2003). “Structure-switching Signaling Aptamers.” J. Am. Chem. Soc. 125:4771-4778
Mochizuki, S (2003). “Measurement of acetylcholine-induced endothelium-derived nitric oxide in aorta using a newly developed catheter-type nitric oxide sensor.” Biochem Biophys Res Commun 306(2): 505-8.
Morales, D. L. (2000) “Arginine vasopressin in the treatment of 50 patients with postcardiotomy vasodilatory shock.” Ann. Thorac. Surg. 69:102-106
Morales, D. L. (1999). “Reversal by vasopressin of intractable hypotension in the late phase of hemorrhagic shock.” Circulation 100(3): 226-9.
Oliver, J (1978). “The effect of altered sodium balance upon renal vascular reactivity to angiotensin II and norepinephrine in the dog. Mechanism of variation in angiotensin responses.” J Clin Invest 61(3): 610-23.
Oliver, J (1980). “Basal norepinephrine overflow into the renal vein: effect of renal nerve stimulation.” Am J Physiol 239(4): F371-F377.
Robin J K (2003). “Vasopressin deficiency in the syndrome of irreversible shock.” J Trauma 54:52-56
Sachs, H. (1967). “Capacity of the neurohypophysis to release vasopressin.” Endocrinology 81(4); 755-70,
Seo S(1998). “Renin inhibits the vasorelaxation induced by nitosoalbumin.” Biochem. Biophys. Res. Comm. 250:94-98
Shackford, S. R. (1988). “The effect of hemorrhage and resuscitation on serum levels of immunoreactive atrial natriuretic factor.” Ann Surg 207(2): 195-200.
Shimura, K (1994). “Affinity probe capillary electrophoresis; analysis of recombinant human growth hormone with fluorescent labeled antibody fragment.” Anal. Chem. 66:9-15.
Stadlbauer, K. H. (2003). “Vasopressin, but not fluid resuscitation, enhances survival in a liver trauma model with uncontrolled and otherwise lethal hemorrhagic shock in pigs.” Anesthesiology 98(3): 699-704.
Stojanovic M N (2003). “Cross-reactive arrays based on three-way junctions.” J. Am. Chem. Soc. 125:6085-6089
Stojanovic, M N (2002). “Aptamer-based colorimetric sensor for cocaine.” J. Am. Chem. Soc. 124:9678.
Stojanovic M N (2001). “Aptamer-based folding fluorescent sensor for cocaine.”J. Am. Chem. Soc. 123(21):4928-4931
Stojanovic, M N (2001). “Folding oligonucleotide-based sensors for cocaine.” J. Am. Chem. Soc. 123:4938.
Sun, Q (2003) “Low-Dose Vasopressin in the Treatment of Septic Shock in Sheep.” Am. J. Respir. Crit. Care Med. 168:481-486
Tahara, A (1998) Br. J. Pharmacol 125:1463.
Taylor, M. S. (2003). “Altered expression of small-conductance Ca2+-activated K+ (SK3) channels modulates arterial tone and blood pressure.” Circ Res 93(2): 124-31.
Tenakin, T (2002). “Efficacy at O-protein-coupled receptors.” Nature Reviews Drug Discovery I:103-110.
Tuerk C (1990). “Systematic Evolution of Ligands by Exponential Evolution: RNA ligands to bacteriophate T4 polymerase.” Science 249:505-510,
Tung, C.-H. (2003). “A novel newar-infrared fluorescence sensor for detection of thrombin activation in blood.” Chem. Bio. Chem. 3:207-211.
Wang Y (1996). “RNA molecules that specifically and stoichiornetrically bind aminoglycoside antibiotics with high affinities.” Biochemistry 35:12338-46.
Wierenga, W (1991). “The motivation: A top down view, in Integrated Drug Discovery.” Drug News Persp. May: 217-222.
Williams, K. P. (1997). Bioactive and nuclease-resistant L-DNA ligand of vasopressin. Proc. Natl. Acad. Sci. USA 94:11285-11290.
Yamamoto R (2000). Gen. Cell. 5:389-396.)
Yamamura Y (1991). “OPC-21268, an orally effective, nonpeptide vasopressin V1 receptor antagonist.” Science 252:572-574.
Ysewijin-Van Brussel, K. A (1985). Clinical Chemistry 3.1:961.
Zhou, M., (2001). “The small intestine plays an important role in upregulating CGRP during sepsis.” Am J Physiol Read Integr Comp Physiol 280(2): R382-8.

Various publications are cited herein, the contents of which are hereby incorporated by reference in their entireties.

We claim:

1. An in vitro assay for determining the amount of a neuro-humoral factor in a patient sample in an acute care setting comprising
(i) exposing the patient sample to (a) an aptamer that binds to the neuro-humoral factor and (b) a bead which binds to the aptamer, wherein the neuro-humoral factor and the bead compete for binding to the aptamer and binding of the aptamer to the bead takes the aptamer out of solution; and
(ii) measuring, using real time polymerase chain reaction, the amount of aptamer in solution,
wherein the concentration of aptamer measured by polymerase chain reaction is directly proportional to the amount of neuro-humoral factor in the patient sample.

2. The assay of claim 1 where the bead comprises, at its surface, bound neurohumoral factor.

3. The assay of claim 1 where the bead comprises, at its surface, bound oligonucleotide, where said oligonucleotide is complementary to at least a portion of the aptamer.

4. An in vitro assay for determining the amount of a neuro-humoral factor in a patient sample in an acute care setting comprising
(i) exposing the patient sample to an aptamer bound to a chromatographic support matrix, where the aptamer binds to the neuro-humoral factor, and
(ii) measuring the amount of neuro-humoral factor which binds to the aptamer.

5. The assay of claim 4 wherein the amount of neuro-humoral factor which binds to the aptamer is measured by determining the amount of a detectably labeled antibody specific for the neuro-humoral factor which binds to the neuro-humoral factor retained on the support matrix.

6. The assay of claim 4 wherein the amount of neuro-humoral factor which binds to the aptamer is measured by determining the amount of detectably labeled neuro-humoral factor, which was pre-bound to the aptamer, is displaced from the support matrix.

7. An in vitro assay for determining the amount of a neuro-humoral factor in a patient sample in an acute care setting comprising
(i) exposing the patient sample to an aptamer that binds to the neuro-humoral factor;
(ii) separating, by capillary electrophoresis, free versus bound aptamers; and
(iii) measuring the amount of free and/or bound aptamer,
wherein the amount of neuro-humoral factor in the patient sample is directly proportional to the amount of bound aptamer and indirectly proportional to the amount of unbound aptamer.

8. An in vitro assay for determining the amount of a neuro-humoral factor in a patient sample in an acute care setting comprising
(i) exposing the patient sample to microspheres linked to aptamer pre-bound to detectably labeled neuro-humoral factor, where the neuro-humoral factor in the patient sample can competitively displace the detectably labeled neuro-humoral factor bound to the aptamer; and
(ii) measuring the amount of displaced detectably labeled neuro-humoral factor using a fiber-optic biosensor system.

9. A homogeneous in vitro assay for determining the amount of a neuro-humoral factor in a patient sample in an acute care setting comprising
(i) exposing the patient sample to a labeled aptamer, wherein binding of the neuro-humoral factor to the aptamer changes the detectable signal generated by the label; and
(ii) using the change in detectable signal to determine the amount of neuro-humoral factor present in the patient sample.

10. The assay of claim 9 wherein the label of the aptamer is bound detectably labeled neuro-humoral factor, wherein neuro-humoral factor in the sample competes with the detectably labeled neurohumoral factor for binding to the aptamer.

11. The assay of claim 9 wherein the aptamer is bound to a fluorophore, the fluorescence of which increases upon binding to neuro-humoral factor.

12. The assay of claim 9 wherein the aptamer is bound to a fluorescence donor and a fluorescence acceptor, and the fluorescent signal increases upon binding to neuro-humoral factor.

13. The assay of claim 9 wherein the aptamer is a heteroaptamer comprising a first and second component wherein the first component is bound to a fluorescence donor and the second component is bound to a fluorescence acceptor, and wherein the components assemble together upon binding to neuro-humoral factor, resulting in a change in fluorescent signal.

14. The assay of claim 9 wherein the aptamer is bound to a luminescent dye, the signal of which decreases upon binding to neuro-humoral factor.

15. An in vitro assay for determining the amount of a neuro-humoral factor in a patient sample in an acute care setting comprising
(i) exposing the patient sample to a heteromeric aptamer, a complementary linking oligonucleotide, and a ligase, wherein binding of the neuro-humoral factor results in assembly of the aptamer and the linking oligonucleotide to produce a ligated product; and
(ii) using real-time polymerase chain reaction to detect and measure the amount of ligated product;
wherein the amount of ligated product is directly proportional to the amount of neuro-humoral factor present.

16. The assay of claim 1, wherein the neuro-humoral factor is vasopressin.

17. The assay of claim 1, wherein the neuro-humoral factor is selected from the group consisting of vasopressin, norepinephrine, epinephrine, renin, endothelin peptide, atrial natriuretic peptide, and brain natriuretic peptide.

18. A method of detecting whether a patient in an acute care setting is at risk for a decrease in blood pressure to undesirable levels, comprising using an assay according to claim 1 to determine the amount of neuro-humoral factor in the
patient, whereby a change in the level of neuro-humoral factor of at least about 20 percent relative to normal or acute shock levels of the neuro-humoral factor is predictive of a decrease in blood pressure to undesirable levels.
